(19) United States
(12) Patent Application Publication
(10) Pub. No.: US 2021/0338452 A1
(43) Pub. Date: Nov. 4, 2021

(54) INTEROSSEOUS CAGE

(71) Applicant: Hassan RAZIAN, L'HAY les Roses (FR)

(72) Inventor: Hassan RAZIAN, L'HAY les Roses (FR)

(21) Appl. No.: 17/268,911

(22) PCT Filed: Aug. 13, 2019
(86) PCT No.: PCT/FR2019/000133

§ 371 Date: Feb. 16, 2021

(30) Foreign Application Priority Data
Aug. 17, 2018 (FR) 18 70935

(51) Int. Cl. A61F 2/44; A61F 2/30

(52) U.S. Cl. CPC A61F 2/446; A61F 2/30771; A61F 2002/30125; A61F 2002/30011; A61F 2002/30014; A61F 2310/00353; A61F 2002/30772; A61F 2002/30841; A61F 2002/3092; A61F 2002/3093; A61F 2310/00023; A61F 2002/30593

(57) ABSTRACT

Disclosed are interosseous cages interposed between two bones. The cage includes at least one body with two bearing faces each bearing face being configured so as to come into contact with the two bones, the body being produced from a first material having a given strength, a through cavity produced in the body opening onto the two faces and an insert defined between two end faces and having a cross section which is at most equal to the cross section of the through cavity, in a manner such that the insert is slid into the cavity, this insert furthermore being configured in a manner such that its end faces are respectively a continuation of the two faces when the insert is in the cavity, the insert being produced from a second material which is stronger than the first material. Application primarily to the production of intervertebral cages.

### BACKGROUND OF THE INVENTION

### Field of the Invention

The present invention relates to interosseous cages, namely cages that can be implanted between two portions of bone with a view to firmly securing them together to encourage their consolidation by osteosynthesis, and which have particularly advantageous applications, albeit not exclusive applications, as intervertebral cages.

### Description of the Related Art

Many interosseous cages of this type are already known. They are essentially composed of a solid body, for example produced from a metal, comprising a through cavity in which a bone graft or bone substitute is placed which can be used to encourage growth of the bone and therefore joining of the two portions of bone by osteosynthesis.

Interosseous cages also exist in which the solid body is produced from a material which is less strong than the metal, for example that which is known to the person skilled in the art as PEEK (polyether ether ketone).

An interosseous cage as defined above provides good results as regards orthopaedic therapy, but may suffer from disadvantages when it is interposed between two portions of bone which could be subjected to relatively large push forces. This is the case which might arise for a cage interposed between two vertebrae in order to replace a damaged intervertebral disc of the spinal column of a patient who is still relatively young and physically strong. In fact, PEEK is not a strong enough material to be able to resist push forces permanently,

Furthermore, PEEK, as defined above, is a material which has the capability of fusing with the bone medium. For this reason, the cage could become at least partially embedded in the bone and no longer act as a wedge, i.e. a spacer, in order to maintain the two portions of bone at the proper distance apart which is desired by the clinician.

Bone cages are already known, such as those described and illustrated in EP 1 414 376 and US 2015/073556. However, those cages in accordance with this prior art neither directly nor indirectly propose a solution to the problem which is mentioned above.

### SUMMARY OF THE INVENTION

The objective of the present invention is therefore to provide an interosseous cage which, even though it is simple and inexpensive in construction, overcomes the disadvantages mentioned above of similar cages of the prior art.

More precisely, the present invention concerns an interosseous cage which is capable of being interposed between two portions of bone with a view to joining them by osteosynthesis, which has at least one of the disclosed features.

### BRIEF DESCRIPTION OF THE DRAWINGS

Other features and advantages of the present invention will become apparent from the following description which is given by way of illustration, which is not in any way limiting in scope, made with reference to the accompanying drawings in which:

FIGS. 1 and 2 show, in two sectional views, a highly diagrammatic representation of an embodiment of the interosseous cage in accordance with the invention; FIG. 1 is a section with reference I-I in FIG. 2, and FIG. 2 is a section with reference II-II in FIG. 1, and

FIG. 3 is an isometric projection of an embodiment of an essential element of the subject matter of the invention, namely the insert which is shown in FIGS. 1 and 2.

### DESCRIPTION OF THE PREFERRED EMBODIMENTS

First of all, it should be pointed out that the term “solid material” as used in the context of the present invention should be understood to mean a material which has a structure, a quality which enables it to resist wear and/or the risk of breakage and/or tearing and/or rupture, etc, under the action of push forces of a given intensity.

For this reason, for the definition of the present invention, mention shall be made in the description and in the accompanying claims of at least a pairing of materials wherein one is stronger than the other in accordance with the above definition.

With reference to the accompanying figures, the present invention concerns an interosseous cage which is capable of being interposed between two portions of bone O1, O2 with a view to joining them by osteosynthesis.

In accordance with this invention, the cage comprises at least one body 10 delimited by two opposed bearing faces 11, 12, each bearing face being configured so as to be capable of coming into contact with a corresponding face of one of the two portions of bone, this body 10 being produced from a first material having a given strength.

The cage also comprises a through cavity 20 produced in the body 10 and opening onto the two bearing faces 11, 12, and an insert 30 defined between two end faces 31, 32 and having a cross section which is at most equal to the cross section of the through cavity 20 in a manner such that this insert is capable of being slid into the cavity.

This insert is also configured in a manner such that its end faces are respectively substantially a continuation of the two bearing faces 11, 12 of the body 10 when the insert 30 is in the through cavity 20.

The insert is produced from a second material which is stronger than the first material.

As mentioned above, the insert 30 is configured in a manner such that its end faces 31, 32 are respectively substantially a continuation of the two bearing faces 11, 12 of the body 10 when the insert 30 is in the through cavity 20. However, in accordance with a further embodiment, this insert is configured in a manner such that its end faces 31, 32 are respectively slightly set back from the two bearing faces 11, 12 of the body 10 when the insert 30 is in the through cavity 20.

This setback may be of advantage in allowing the passage of a cutting blade in order to cut the trabeculae which, after a certain time following implantation of a cage of this type, connect the cancellous bone to the insert 30. This intervention may, for example, be carried out in the case of repeating surgery after revision of the bone structure.

The fact that these setbacks as defined above also correspond to slots or the like in the body 10 is also advantageous; they allow the passage of cutting blades, which can access these setbacks without difficulty.

This above feature is highly advantageous because, during the insertion of the cage between the two portions of bone O1, O2, it is then certain that at least a portion of the two end faces of the insert will not protrude from the bearing faces 11, 12 of the body, and that the end faces of the insert will not damage the bony faces of the portions of bone O1, O2 against which the two opposed faces of the cage (i.e. the bearing faces 11, 12 of the body 10 and the end faces 31, 32 of the insert 30) will come to bear.

In accordance with a preferred feature, the insert 30 has a porous structure, yet more advantageously a porous honeycomb structure.

More preferably again, the honeycomb structure comprises at least one continuous channel 35 opening onto the two ends of the insert.

In accordance with a preferred feature, the cross section of the insert 30 may be equal to that of the cavity. In this case, the insert 30 and the body 10 may be produced in a single piece by being manufactured by moulding one part with or on the other.

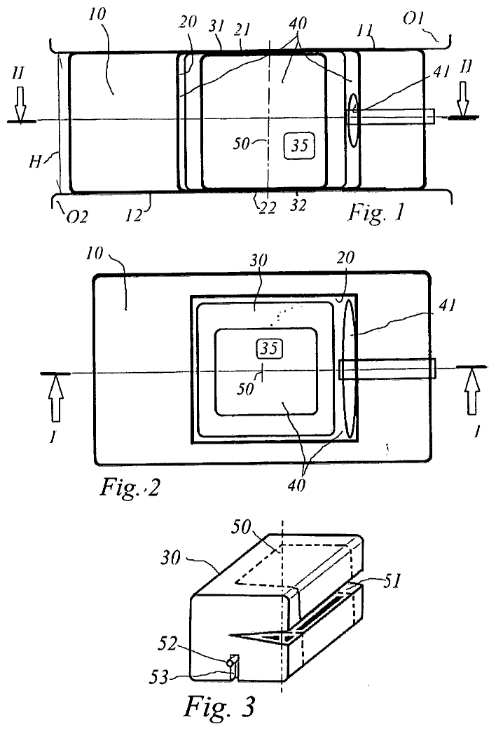

In accordance with a particularly advantageous embodiment, as illustrated in FIG. 3 and with reference to the embodiment in accordance with FIGS. 1 and 2, the disposition of the insert 30 is such that it has an increased elasticity along a direction 50 which is substantially perpendicular to the two opposed bearing faces 11, 12 in a manner such that when this insert is in a state such that it is compressed along this direction 50, its height defined along this direction is at most equal to the distance separating the opposed bearing faces 11, 12 and that when it is in the relaxed state, its height is greater than that distance.

With the feature described above, the cage in accordance with the invention may be introduced, for example between two vertebrae O1 and O2, by means of an instrument which is known in the surgical field, by keeping the insert 30 in its compressed state, which facilitates implantation of the cage between these two vertebrae.

After the cage has been put in position and the instrument removed, the insert relaxes and therefore emerges very slightly from the through cavity 20. In this relaxed state, the two end faces 31, 32 of the insert, which may optionally include anchoring pins or the like, come into positive contact with the two bone faces and bear firmly against them, preventing the cage from moving and/or migrating after it has been implanted even more securely.

This increased elasticity of the insert 30 as defined above may be obtained in a number of ways. However, the simplest and the most industrial manner is obtained by producing a slot 51 in the insert, in a plane substantially parallel to the two end faces 31, 32, as illustrated in FIG. 3.

Preferably, the cage furthermore comprises means for defining indexing of the position of the insert 30 in the through cavity 20, in order to uniquely define the position of the insert in this cavity, in order to facilitate the clinician's preparatory work.

These means for indexing the position of the insert in the through cavity may also be produced in different manners. As an example, as illustrated in FIG. 3, they may be constituted by a pin 52 or the like which is secured to the body 10 and a groove of a predetermined length 53 produced in the insert 30 (or vice versa: a pin which is secured to the insert and a groove produced in the body 10). In this manner, when the interposed is introduced into the cavity 20, its final position is well defined when the pin 52 comes into abutment against the end wall of the groove 53.

However, these indexing means may also, for example, be constituted by a tapered fit between the insert and the cavity. In this case, the insert 30 and the cavity 20 each have at least one tapered portion which is complementary to the other. These two portions with a tapered shape uniquely limit the fit of the insert into the cavity obtained when the two tapered surfaces come into contact and align with each other.

A combination of the two embodiments described above may also be envisaged.

It should be pointed out that in FIG. 3, the insert shown in solid lines is substantially in the form of a parallelepipedal rectangle, but it may take other shapes, for example it could be U-shaped, as shown in dashed lines on the same figure.

Furthermore, the cross section of the insert 30 may be smaller than the cross section of the cavity 20 in a manner such that, as illustrated in FIGS. 1 and 2, between the insert and the wall of the cavity, there is at least one gap 40 determined so that it is capable of receiving at least one of the following elements: an anchoring strip 41, a bone graft, a bone substitute, or a combination of at least two of these elements.

It should also be pointed out that the anchoring strip may be mounted integrally with the insert 30 or the body 10. FIGS. 1 and 2 show, by way of non-limiting example, the case of a strip which is pivotably mounted with respect to the body 10. A rotatable mount with respect to the insert 30 would not cause any difficulties to the person skilled in the art and for this reason, this embodiment has not been specifically illustrated.

In accordance with a highly preferable embodiment, the first material is produced from PEEK and the second material is highly advantageously selected from the following products:

titanium, titanium mesh, porous titanium, Trabecular Titanium™, a combination of at least two of these products.

It should be pointed out that although the body 10 and the through cavity 20 have both been shown in the form of parallelepipedal rectangles, they may have any other shape, in particular shapes that are known in the prior art. This specific shape as illustrated was used for the purposes of simplification of the drawings and for facilitating comprehension of the present invention.

In this regard, in accordance with a possible feature of the invention, the through cavity 20 is oval or the like in shape. This latter embodiment is interesting because it means that a through cavity 20 can be obtained which has a larger volume than that of a cylindrical cavity, without in any way reducing the area of the two opposed bearing faces 11, 12 of the body 10, or modifying the two end faces 31, 32 of the insert 30. In contrast, it can, for example, be used to house a large quantity of bone substitute or the like in order to encourage osteosynthesis.

With a cage in accordance with the invention, the distance between the two portions of bone will be preserved and maintained, even during the course of the fusion of PEEK with the bone medium, because the insert ensures permanent support throughout fusion.

1. An interosseous cage which is capable of being interposed between two portions of bone with a view to joining them by osteosynthesis, comprising at least:
a body delimited by two opposed bearing faces, each bearing face being configured so as to be capable of coming into contact with a corresponding face of one of the two portions of bone, said body being produced from a first material having a given strength, and
a through cavity produced in said body and opening onto the two bearing faces,
further comprising an insert defined between two end faces and having a cross section which is at most equal to the cross section of said through cavity in a manner such that said insert is capable of being slid into the cavity, this insert furthermore being configured in a manner such that the end faces are respectively substantially a continuation of the two bearing faces of said body when said insert is in the through cavity, said insert being produced from a second material which is stronger than the first material.

2. The cage as claimed in claim 1, wherein said insert has a porous structure.

3. The cage as claimed in claim 2, wherein said insert has a porous honeycomb structure.

4. The cage as claimed in claim 3, wherein the honeycomb structure comprises at least one continuous channel opening onto the two ends of said insert.

5. The cage as claimed in claim 1, wherein the cross section of said insert is less than the cross section of said cavity in a manner such as to leave, between said insert and the wall of said cavity, at least one gap capable of receiving at least one of the following elements: an anchoring strip, a bone graft, a bone substitute, or a combination of at least two of these elements.

6. The cage as claimed in claim 1, wherein said first material is produced from PEEK and wherein the second material is selected from the group consisting of: titanium, titanium mesh, porous titanium, Trabecular Titanium™, and a combination of at least two of these products.

7. The cage as claimed in claim 1, wherein said insert is configured in a manner such that the end faces are respectively slightly set back from the two bearing faces of the body when the insert is in the through cavity.

8-11. (canceled)

12. An interosseous cage which is capable of being interposed between two portions of bone with a view to joining them by osteosynthesis, comprising at least:
a body delimited by two opposed bearing faces, each bearing face being configured so as to be capable of coming into contact with a corresponding face of one of the two portions of bone,
a through cavity produced in said body and opening onto the two bearing faces, and
an insert defined between two end faces and having a cross section which is at most equal to the cross section of said through cavity in a manner such that said insert is capable of being slid into the cavity,
wherein this insert is furthermore configured in a manner such that its end faces are respectively slightly set back from the two bearing faces of the body when the insert is in the through cavity, said insert having a porous structure.

13. The cage as claimed in claim 12, wherein said insert has a porous honeycomb structure.

14. The cage as claimed in 13, wherein the honeycomb structure comprises at least one continuous channel opening onto the two ends of said insert.

15. The cage as claimed in claim 12, wherein the cross section of said insert is less than the cross section of said cavity in a manner such as to leave, between said insert and the wall of said cavity, at least one gap capable of receiving at least one of the following elements: an anchoring strip, a bone graft, a bone substitute, a combination of at least two of these elements.

16. The cage as claimed in claim 12, wherein said through cavity is oval in shape.

17. The cage as claimed in claim 12, further comprising means for defining indexing of the position of said insert in said through cavity.

18. The cage as claimed in claim 2, wherein the cross section of said insert is less than the cross section of said cavity in a manner such as to leave, between said insert and the wall of said cavity, at least one gap capable of receiving at least one of the following elements: an anchoring strip, a bone graft, a bone substitute, or a combination of at least two of these elements.

19. The cage as claimed in claim 3, wherein the cross section of said insert is less than the cross section of said cavity in a manner such as to leave, between said insert and the wall of said cavity, at least one gap capable of receiving at least one of the following elements: an anchoring strip, a bone graft, a bone substitute, or a combination of at least two of these elements.

20. The cage as claimed in claim 4, wherein the cross section of said insert is less than the cross section of said cavity in a manner such as to leave, between said insert and the wall of said cavity, at least one gap capable of receiving at least one of the following elements: an anchoring strip, a bone graft, a bone substitute, or a combination of at least two of these elements.

21. The cage as claimed in claim 2, wherein said first material is produced from PEEK and wherein the second material is selected from the following products: titanium, titanium mesh, porous titanium, Trabecular Titanium™, a combination of at least two of these products.

22. The cage as claimed in claim 3, wherein said first material is produced from PEEK and wherein the second material is selected from the following products: titanium, titanium mesh, porous titanium, Trabecular Titanium™, a combination of at least two of these products.

23. The cage as claimed in claim 4, wherein said first material is produced from PEEK and wherein t the second material is selected from the following products: titanium, titanium mesh, porous titanium, Trabecular Titanium™, a combination of at least two of these products.

24. The cage as claimed in claim 5, wherein said first material is produced from PEEK and wherein the second material is selected from the following products: titanium, titanium mesh, porous titanium, Trabecular Titanium™, a combination of at least two of these products.
